(19) United States
(12) Patent Application Publication
(10) Pub. No.: US 2006/0287625 A1
(43) Pub. Date: Dec. 21, 2006

(54) Total contact thoraco-lumbosacral spinal orthosis

(76) Inventor: Isabelle Rauch, New York City, NY (US)

Correspondence Address: MILES & STOCKBRIDGE PC, 1751 PINNACLE DRIVE, SUITE 500, MCLEAN, VA, 22102-3833, US

(21) Appl. No.: 10/265,287

(22) Filed: Oct. 4, 2002

Related U.S. Application Data
(60) Provisional application No. 60/327,393, filed on Oct. 5, 2001.

(51) Int. Cl. A61F 5/00

(52) U.S. Cl. 602/19; 602/18

(57) ABSTRACT

The present invention is an anterior opening one piece total contact spinal orthotic device with an anterior attached upper body attachment that combines a three point pressure system for spinal hyperextension with an abdominal hydraulic pressure support system. This is accomplished by including a longitudinal pressure bar in the medial sagittal plane that attaches to the lower anterior face of an overlap style total contact module and at the upper end to a chest plate. The chest plate is either a sternal plate or a pectoral pad bridge unit. The medial posterior point proximal to T-10 or there about is anchored to the pressure bar through the upper structure of the total contact module. A variety of additional attachments, features and options are accommodated.

### FIELD OF THE INVENTION

This invention relates to orthotic devices. More specifically, this invention relates to orthotic devices that provide rigid frame spinal bracing of upper body musculoskelatal injury or disease occurring in the thoracic, lumbar or sacral region to the sacral pelvic region.

### BACKGROUND

The normal anatomy of the spine is usually described by dividing up the spine into 4 major sections: the cervical, the thoracic, the lumbar, and the sacral spines. Each section is made up of individual bones called vertebrae. There usually are 7 cervical vertebrae, 12 thoracic vertebrae, and 5 lumbar vertebrae. The sacrum, below the lumbar vertebrae, is the spinal part of the pelvis and is considered to be 5 vertebrae that have fused into one bone.

An individual vertebra is made up of several parts. The body of the vertebra is the primary area of weight bearing and provides a resting place for the fibrous discs which separate each of the vertebrae. The lamina covers the spinal canal, the large hole in the center of the vertebra through which the spinal nerves pass. The spinous process is the bone you can feel when running your hands down your back. The paired transverse processes are oriented 90 degrees to the spinous process and provide attachment for back muscles. There are four facet joints associated with each vertebra. A pair that face upward and another pair that face downward. These interlock with the adjacent vertebrae and provide stability and integrity to the spine.

The vertebrae are separated by intervertebral discs which act as cushions between the bones. Each disc is made up of two parts. The hard, tough outer layer called the annulus surrounds a mushy, moist center termed the nucleus. When a disc herniates or ruptures, the soft nucleus spurts out through a tear in the annulus, and can compress a nerve root. The nucleus can squirt out on either side of the disc or in some cases both sides. The amount of pain associated with a disc rupture often depends upon the amount of nucleus that breaks through the annulus, and whether it compresses a nerve. In order to relieve the associated pain it is sometimes necessary to temporarily hyperextend the spine by using some type of external orthosis.

Other pathological conditions also require temporary or permanent orthotic bracing such as: lordosis, an exaggerated forward curvature of the lumbar spine; scoliosis, a lateral curvature of the spine; kyphosis, an angular curvature of the spine, usually in the thoracic region, combinations of these with each other or rotation, as well as a variety of spinal fractures and soft tissue injuries.

The fact that spinal orthotic devices (devices intended for augmentation, immobilization and/or isolation of spinal structure in patients with acute and chronic spinal injury) are available in such a large variety, evidences both the complexity of the problem, which the devices are intended to address, as well as the need for solutions specific to each patient's unique size, musculo-skeletal configuration, and pathology. The clinician's armamentarium of orthotics presently available includes such familiar choices as the Jewitt hyperextension orthosis (FIG. PA9), and the Knight-Taylor spinal orthosis (FIG. PA5), as well as some newer total contact bivalve and body jacket designs.

Referring to FIG. PA1 an orthosis of the old canvas construction is incapable of transmitting referred forces through attachments, complicated to doff, bulky, difficult for patients to tighten sufficiently to be effective, and cannot easily provide the rigidity of support needed. Referring to FIG. PA-2, a compression support is only that, capable of inducing intra-abdominal hydraulic pressure, although it has attempted to solve the difficulty of tension adjustment by a unique strap tensioning system.

Referring collectively to FIGS. PA-3, PA-5, PA-6, and PA-7, multiple orthotics have attempted to address the classic problems of instability and inability to support referred force attachments of the soft orthoses by introducing panels and structural frames made of semi-rigid semi-flexible plastics. Here each orthotic has such a structure but additional strapping is still requires and the addition of the support panels adds extra weight to the already bulky cloth devices.

Referring to FIGS. PA-8 and PA-9, the CASH, and Jewett orthoses approach the problem by simple cantilever approaches. These devices use what is known as a three point system to induce a hyperextension in the spine around a posterior plate anchored by straps to a bridge connecting two anterior pressure plates at the chest and lower abdomen. The approach produces a light, minimally structured device but ignores the augmenting advantages of intra-abdominal hydraulic pressure stiffening. Consequently, higher forces are required to accomplish the hyperextension. Additionally, this approach concentrates all the posture correcting forces into three contact points. This force-concentrating feature results in a brace that is often uncomfortable and sometimes painful. Also, these devices having little surface contact have poor ability to provide reliable alignment stability features.

Referring collectively to FIGS. PA-4 and PA-10a-d, the bivalves and body jackets represent the current state of the art in orthotics. These devices are made almost entirely of two layered sheets of semi-rigid semi-flexible polymer and a resilient foam lining. These orthotics can be heat molded to the patient and provide both strength to support attachments as well as more comfortable hydraulic pressurization.

The bivalves (FIGS. PA-4 and PA-10c) are of two sheets, one each anterior and posterior, that are molded to fit the patient and overlap laterally. Straps are required on both sides to tighten the two plates toward each other stabilizing its position according to its molded shape, as well as increase intra-abdominal pressure. The smooth anterior and posterior surfaces are rigid enough to support either anterior or posterior attachments, often without the usual requirement of additional strapping on the attachment itself. The problematic drawbacks, are the heaviness of the devices (often 6-15 lbs), and the difficulties in doffing and tightening all the many adjustment straps evenly to attain efficacious snugness and alignment.

The Body Jackets FIGS. PA-10a, b, and d. present a one piece molded sheet with a small longitudinal opening in either the anterior or posterior or with an overlap in either the anterior or posterior. They are lighter and easier to adjust than the bivalves. The patient doffs the jacket by springing it open and sliding through the opening, once in place it is tightened. If a posterior attachment is desired then an anterior opening jacket is required and the doffing adjustment process is straight forward. However, if an anterior attachment is prescribed, the doffing/adjustment process is very difficult without the help of a second party. Anterior attachments with anterior opening body jackets are not available. Additionally, the longitudinal and tortional skew stability of the jacket at the anterior or posterior opening is dependant solely on the straps bridging the opening, creating an unstable location for an attachment and making lateral stability less efficacious.

Referring specifically to FIG. PA10d the anterior opening anterior overlap body jacket utilizes the ease of doffing/adjustment usual for an anterior opening body jacket along with the light weight and stabilizing rigidity of a hard shell. If worn tightly it induces a good reliable intra-abdominal hydraulic pressure. It is considered useful for addition of a Knight Taylor extension but because of the anterior location of the overlap flaps this orthosis is not thought useful for the addition of anterior extensions. Also it is disfavored for anterior attachments because of the perceived difficulty of maintaining medial alignment, in the face of frequent size adjustment necessary accommodate ongoing changes in the patient body size and shape, for an attachment that is located on a closure flap. Finally, the smooth outer surface of the jacket presents a decreased frictional component, so that the overlap contributes far less that would be expected to longitudinal and tortional skewing stability at the juncture. It would therefore be highly advantageous to have an anterior attachment capable body jacket that also features the weight, doffing and adjustment features of an anterior opening or anterior overlap body jacket.

### OBJECTS AND SUMMARY OF THE INVENTION

It is an object of the present invention to overcome the drawbacks of the prior art. In this regard, it is an object of the present invention to provide an anterior attachment capable body jacket that also features the light-weight, ease of doffing and ease of adjustment features of an anterior opening or anterior overlap body jacket.

It is another object of the present invention to provide a spinal orthotic, including; a total contact module constructed of a single piece of a semi-rigid semi-resilient material, the total contact module having an anterior overlap, a longitudinal pressure bar positioned anterior to the total contact module, the longitudinal pressure bar spanning first and second posteriorly directed force vectors, a module attachment at the first posteriorly directed force vector, the module attachment attaching a module attachment end of the longitudinal pressure bar to the total contact module, a chest plate attachment at the second posteriorly directed force vector, the chest plate attachment attaching a chest plate attachment end of the longitudinal pressure bar to a chest plate, at least one adjustable strap on the module spanning the anterior overlap, at least one of the at least one adjustable strap being capable of increasing the anterior overlap to reduce a circumferential size of the total contact module thereby increasing abdominal hydraulic pressure, at least one of the at least one adjustable strap being capable of inducing an anteriorly directed third force parallel with, between, and diametrically opposite the first and second forces by connecting a tenth thoracic vertebrae proximal region on a posterior side of the total contact module with a posterior pressure directing anchor point on the longitudinal pressure bar between the chest plate attachment end and the module attachment end; and, the first, second, and third forces collectively forming a three point pressure system having a spinal extension directing effect.

It is another object of the present invention to provide an orthosis, including, a one piece abdominal enclosure having an anterior overlap, the one piece abdominal enclosure capable of exerting an abdominal hydraulic pressure; and a chest plate attached to the anterior overlap by a pressure bar whereby the one piece abdominal enclosure, the chest plate, and the pressure bar collectively in concert form a three point spinal hyperextension pressure system.

According to an embodiment of the present invention there is provided a spinal orthosis featuring a semi-rigid semi-resilient material being either a homogeneous material or a laminate, the laminate having at least one of a shell and a liner, the shell at least partially having at least one layer of at least one of a clothing contact surface material, a core, a stiffener, and a strengthening material; and, the liner at least partially having at least one layer of at least one of a resilient cushion and a dermal contact surface layer.

According to an embodiment of the present invention there is provided a spinal orthosis also featuring a semi-rigid semi-resilient material being either a homogeneous material or a laminate, the laminate having at least one of a shell and a liner, the shell at least partially having at least one layer of at least one of a clothing contact surface material, a core, a stiffener, and a strengthening material; and, the liner at least partially having at least one layer of at least one of a resilient cushion and a dermal contact surface layer. But wherein; the clothing contact surface material is selected from the group consisting of a plastic, a metal, an alloy, a cloth, a leather, a rubber, a polyethylene, a polypropylene, a polyvinylchloride, a polybuterate, a polystyrene, a polycarbonate, and an aluminum, the core is selected from the group consisting of a plastic, a metal, an alloy, a cloth, a leather, a rubber, a polyethylene, a polypropylene, a polyvinylchloride, a polybuterate, a polystyrene, a polycarbonate, and an aluminum, the strengthening material is selected from the group consisting of a plastic, a metal, an alloy, carbon fibers, glass fibers, plastic fibers, a cloth, a leather, a rubber, a polyethylene, a polypropylene, a polyvinylchloride, a polybuterate, a polystyrene, a polycarbonate, and an aluminum, the stiffener is selected from the group consisting of a plastic, a metal, an alloy, a cloth, a leather, a rubber, a polyethylene, a polypropylene, a polyvinylchloride, a polybuterate, a polystyrene, a polycarbonate, and an aluminum, the resilient cushion is selected from the group consisting of a plastic foam, a cloth, a leather, a rubber foam, a polyethylene foam, a polypropylene foam, a polyvinylchloride foam, a polybuterate foam; and the dermal contact surface material is selected from the group consisting of a plastic, a cloth, a leather, a rubber, a polyethylene, a polypropylene, a polyvinylchloride, a polybuterate, a polystyrene, and a polycarbonate.

According to another embodiment of the present invention there is provided a spinal orthosis featuring the overlap includes at least one inner flap and at least one outer flap, both the inner and outer flaps extend an essentially equivalent distance past a sagittal anterior-posterior midplane; and, the module attachment end of the longitudinal pressure bar attaches to one of the at least one outer flap at the sagittal anterior-posterior midplane.

According to yet another embodiment of the present invention there is provided a spinal orthosis featuring the overlap to include at least one inner flap and at least one outer flap, the inner flap essentially extends completely across an anterior side of the total contact module; and, the outer flap extends a sufficient distance past a sagittal anterior-posterior midplane to allow the module attachment end of the longitudinal pressure bar to attach to one of the at least one outer flap at the sagittal anterior-posterior midplane.

According to yet another embodiment of the present invention there is provided a spinal orthosis featuring the overlap to include at least one inner flap and at least one outer flap, the inner flap essentially extends completely across an anterior side of the total contact module, the outer flap extends a sufficient distance across an anterior side to create the overlap; and, the module attachment end of the longitudinal pressure bar attaches to one of the at least one inner flap at a sagittal anterior-posterior midplane.

According to yet another embodiment of the present invention there is provided a spinal orthosis featuring the overlap to include first and second circumferential end margins of the single piece divided into multiple overlappable flap sections serially positioned, with each touching the next, along each lengths of the first and second circumferential end margins, each overlappable flap section of the first circumferential end margin pairs with a longitudinally aligned one of the overlappable flap sections of the second circumferential end margin as an overlap pair, some the overlappable flap sections of the first circumferential end margin are outer flaps paired with the overlappable flap sections of the second circumferential end margin as inner flaps; and, the remainder of the overlappable flap sections of the first circumferential end margin are inner flaps paired with the overlappable flap sections of the second circumferential end margin as an outer flaps.

According to yet another embodiment of the present invention there is provided a spinal orthosis featuring the chest module being either a sternal plate, or a bridged pectoral pad set.

According to yet another embodiment of the present invention there is provided a spinal orthosis featuring the chest module being either a sternal plate, or a bridged pectoral pad set and wherein at least one of the module attachment and the chest plate attachment is a hinge having an axis of rotation perpendicular to a sagittal anterior-posterior midplane.

According to yet another embodiment of the present invention there is provided a spinal orthosis featuring the chest module being either a sternal plate, or a bridged pectoral pad set and wherein at least one of the module attachment and the chest plate attachment is a hinge having an axis of rotation perpendicular to a sagittal anterior-posterior midplane, the hinge includes at least one additional axis of rotation, each additional axis of rotation is parallel to the axis of rotation, collectively all the axes of rotation defining a compound hinge, the compound hinge including a resilient element; and the resilient element urging the at least one of the module attachment and the chest plate attachment in a posterior direction against the posterior pressure directing anchor point.

According to yet another embodiment of the present invention there is provided a spinal orthosis featuring the overlap to include first and second circumferential end margins of the single piece divided into multiple overlappable flap sections serially positioned, with each touching the next, along each lengths of the first and second circumferential end margins, each overlappable flap section of the first circumferential end margin pairs with a longitudinally aligned one of the overlappable flap sections of the second circumferential end margin as an overlap pair, some the overlappable flap sections of the first circumferential end margin are outer flaps paired with the overlappable flap sections of the second circumferential end margin as inner flaps; and, the remainder of the overlappable flap sections of the first circumferential end margin are inner flaps paired with the overlappable flap sections of the second circumferential end margin as an outer flaps, the longitudinal pressure bar passing between the outer and inner flaps of at least-one the overlap pair, one the outer flap of the at least one the overlap pair having an adjustable latch whereby the one the outer flap can attach to the total contact module and exert a size reducing pressure on the circumferential size; and the one the outer flap is aligned over the posterior pressure directing anchor point and replaces the at least one of the at least one adjustable straps being capable of inducing an anteriorly directed third force.

According to yet another embodiment of the present invention there is provided a spinal orthosis featuring the chest module being either a sternal plate, or a bridged pectoral pad set and wherein at least one of the module attachment and the chest plate attachment is a hinge having an axis of rotation perpendicular to a sagittal anterior-posterior midplane, the hinge includes at least one additional axis of rotation, each additional axis of rotation is parallel to the axis of rotation, collectively all the axes of rotation defining a compound hinge, the compound hinge including a resilient element; and the resilient element urging the at least one of the module attachment and the chest plate attachment in a posterior direction against the posterior pressure directing anchor point, and further including; at least one of the module attachment and the chest plate attachment is a hinge having an axis of rotation perpendicular to a sagittal anterior-posterior midplane, the hinge includes at least one additional axis of rotation, each the additional axis of rotation is parallel to the axis of rotation, collectively all the axes of rotation defining a compound hinge, the compound hinge including a resilient element; and the resilient element urging the at least one of the module attachment and the chest plate attachment in a posterior direction against the posterior pressure directing anchor point.

In summary the present invention is a spinal orthotic device that combines a three point pressure system for spinal hyperextension with an abdominal hydraulic pressure support system. This is accomplished by including a longitudinal pressure bar in the medial sagittal plane that attaches to the lower anterior face of an overlap style total contact module and at the upper end to a chest plate. The chest plate is either a sternal plate or a pectoral pad bridge unit. The medial posterior pressure point proximal to T-10 or there about is a result of the anchor to the pressure bar through the upper structure of the total contact module. A variety of additional attachments, features and options are accommodated.

The above, and other objects, features and advantages of the present invention will become apparent from the following description read in conjunction with the accompanying drawings, in which like reference numerals designate the same elements.

### BRIEF DESCRIPTION OF THE DRAWINGS

FIG. 1A perspective view illustration of an embodiment of the present invention

FIG. 2 A Side elevation view illustration of an embodiment of the present invention.

FIG. 3a A top elevation view illustration of an embodiment of the present invention.

FIG. 3b A top elevation view illustration of another embodiment of the present invention

FIG. 3c An anterior elevation view illustration of another embodiment of the present invention.

FIG. 3d An anterior elevation view illustration of another embodiment of the present invention.

FIG. 3e An anterior close up view illustrating an overlap registration slide of an embodiment of the present invention.

FIG. 4a A top view close up view illustrating a strap tightening ratchet of an embodiment of the present invention.

FIG. 4b A top view close up view illustrating a walking clamp attachment of an embodiment of the present invention.

FIG. 4c A top view close up view illustrating another walling attachment of an embodiment of the present invention.

FIG. 4d A top view elevation illustrating a bladder positioning of an embodiment of the present invention.

FIG. 5a A side view close up illustrating a rotation-axis-translating four pivot resilient hinge of an embodiment of the present invention.

FIG. 5b An anterior view close up illustrating the FIG. 5a rotation-axis-translating four pivot resilient hinge of an embodiment of the present invention.

FIG. 6a A side view close up illustrating a resilience augmentation of a rotation axis translating four pivot resilient hinge of an embodiment of the present invention.

FIG. 6b A side view close up illustrating another resilience augmentation of a rotation axis translating four pivot resilient hinge of an embodiment of the present invention

FIG. 6c A side view close up illustrating yet another resilience augmentation of a rotation axis translating four pivot resilient hinge of an embodiment of the present invention.

FIG. 6d A side view close up illustrating yet another resilience augmentation of a rotation axis translating four pivot resilient hinge of an embodiment of the present invention.

FIG. 6e A side view close up illustrating yet another resilience augmentation of a rotation axis translating four pivot resilient hinge of an embodiment of the present invention.

FIG. 6f A side view close up illustrating yet another resilience augmentation of a rotation axis translating four pivot resilient hinge of an embodiment of the present invention.

FIG. 6g A side view close up illustrating yet another resilience augmentation of a rotation axis translating four pivot resilient hinge of an embodiment of the present invention.

FIG. 6h A side view close up illustrating yet another resilience augmentation of a rotation axis translating four pivot resilient hinge of an embodiment of the present invention.

FIG. 6i A side view close up illustrating yet another resilience augmentation of a rotation axis translating four pivot resilient hinge of an embodiment of the present invention.

FIG. 7a An anterior elevation view illustrating the location of a sternal plate attachment on an embodiment of the present invention.

FIG. 7b An anterior elevation view illustrating the location of a pectoral pad bridge module attachment on an embodiment of the present invention.

FIG. 7c An anterior elevation view illustrating the location of a shoulder pad bridge module attachment on an embodiment of the present invention.

FIG. 7d An anterior elevation view illustrating the location of a double bar cantilever based shoulder pad bridge module attachment on an embodiment of the present invention.

FIG. PA1a A Prior Art Illustration of the anterior view of a soft spinal orthosis.

FIG. PA1b A Prior Art Illustration of the posterior view of a soft spinal orthosis.

FIG. PA2 A Prior Art Illustration of the anterior orthogonal view of a Compression Orthosis.

FIG. PA3 A Prior Art Illustration of the anterior view of an airback spinal orthosis.

FIG. PA4 A Prior Art Illustration of the anterior view of two bivalve orthoses.

FIG. PA5 A Prior Art Illustration of the posterior view of support structure of a Knight Taylor brace.

FIG. PA6 A Prior Art Illustration of the anterior view of a Kydex chairback brace.

FIG. PA7 A Prior Art Illustration of a side view of a chairback brace with a Knight Taylor Extension.

FIG. PA8 A Prior Art Illustration of the anterior view of a CASH orthosis with a Pectoral Pad Bridge type Chest Plate.

FIG. PA9 A Prior Art Illustration of the anterior view of a Jewett Hyperextension Orthosis.

FIG. PA10a A Prior Art Illustration of the anterior view of a posterior opening body jacket.

FIG. PA10b A Prior Art Illustration of the anterior view of an anterior opening body jacket.

FIG. PA10c A Prior Art Illustration of the anterior view of a bivalve body jacket with a Sternal Plate type Chest Plate.

FIG. PA10d A Prior Art Illustration of the anterior view of an anterior opening anterior overlap body jacket.

### DETAILED DESCRIPTION OF THE PREFERRED EMBODIMENT(S)

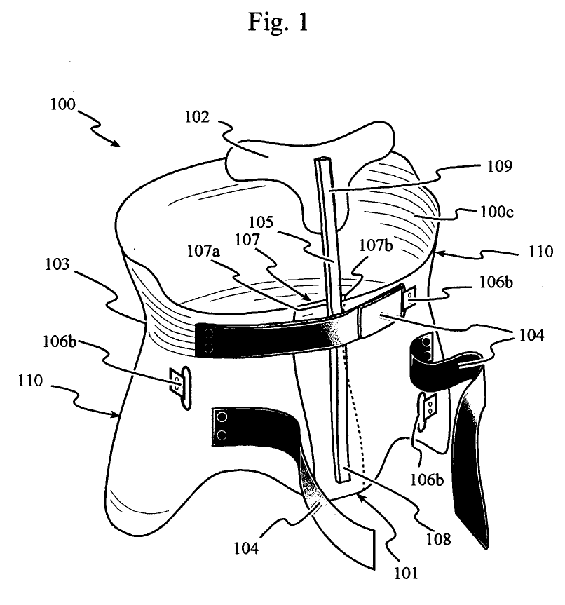

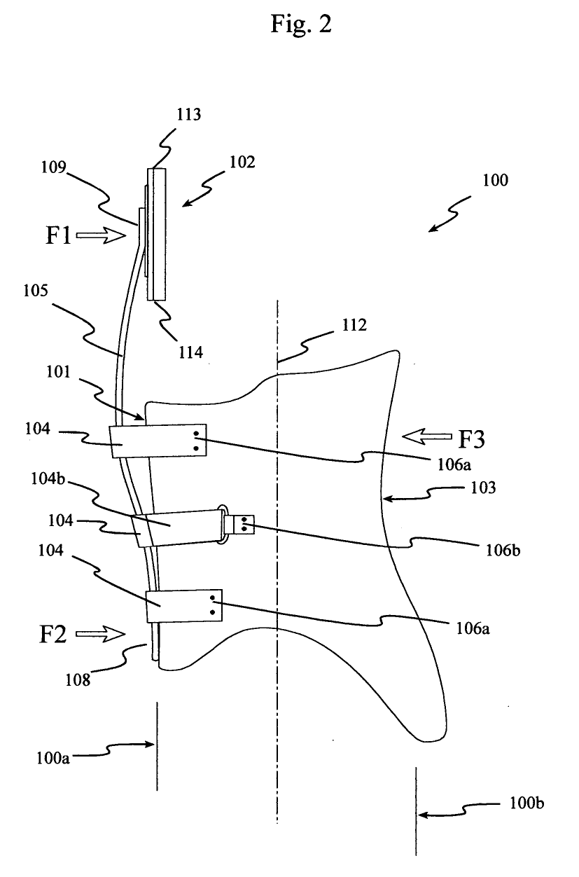

Referring to FIGS. 1 and 2, an embodiment of the present invention, a total contact module 100, having an anterior face 100a, a posterior face 100b, and lateral faces 100c, is connected to a chest plate 102. Total contact module 100, constructed of semi-rigid contourable plastic sheet laminate material of an outer plastic shell 100d layer with a resilient lining 100e layer, wraps around the patient and forms an overlap 107 at anterior face 100a. Overlap 107 allows total contact module 100 to be adjusted to varying levels of tightness by some type of adjustment mechanism. Shown here adjustment is accomplished by multiple opposite facing adjustment straps 104 attached to total contact module 100 on laterally opposite sides of overlap 107 at one side by a non-removable strap attachment 106a and at the opposite side by looping through a strap loop 106b and folding back onto itself Adjustment straps 104 alternate direction with first adjustment strap 104 having non-removable strap attachment 106 on a first lateral side of overlap 107, next adjustment strap 104 having non-removable strap attachment 106a on the lateral side opposite the first, the next opposite that, and so forth.

Some adjustment straps 104 may pass over a stiffly resilient pressure bar 105 to also provide adjustability to the amount of pressure that is exerted by chest plate 102. Pressure bar 105 is attached to total contact module 100 at a module attachment point 108 at a first end and is attached to chest plate 102 at a chest plate attachment point 109. Shown here in this embodiment both module attachment point 108 and chest plate attachment point 109 are rigid attachments that can be made by many means including but not limited to bolts, screws, rivets, adhesives, clamps, molded interlocks, etc. Overlap 107 is shown in this embodiment as a single continuous overlap having an outer overlap flap 107a and an inner overlap flap 107b. Module attachment point 108 attaches preferentially to outer overlap flap 107a.

The walls of total contact module 100 are contoured to exert compression from all sides thereby creating increased hydraulic rigidity in the patients abdominal section. This abdominal hydraulic rigidity increases the support between the pelvis and the thorax thereby reducing the intensity of traditional corrective device pressure needed to achieve the desired result. The corrective action also includes a three-point pressure system, wherein an anterior section 101 of a total contact module 100 provides a posteriorly directed force F1, anterior chest plate 102 provides a second posteriorly directed force F2, and a posterior section 103 of total contact module 100 proximal to the tenth Thoracic Vertebrae level provides an anteriorly directed third force F3. Force F3 opposing, offset and in between the diametrically opposite forces F1 and F2 exerts a spinal extension action. It is recommended that, total contact module 100 is constructed with an initial 15% of lumbar lordosis. Although it is theoretically possible to increase the pressure at F2 by recontouring the metal of pressure bar 105 to apply further pressure for a greater hyperextension moment, patients cannot usually tolerate high forces in this area. Thus, it is orthotically preferable to start by introducing an internal force F3 pressure bolster attached with Velcro to the interior side of posterior section 103, proximal to the T10 area.

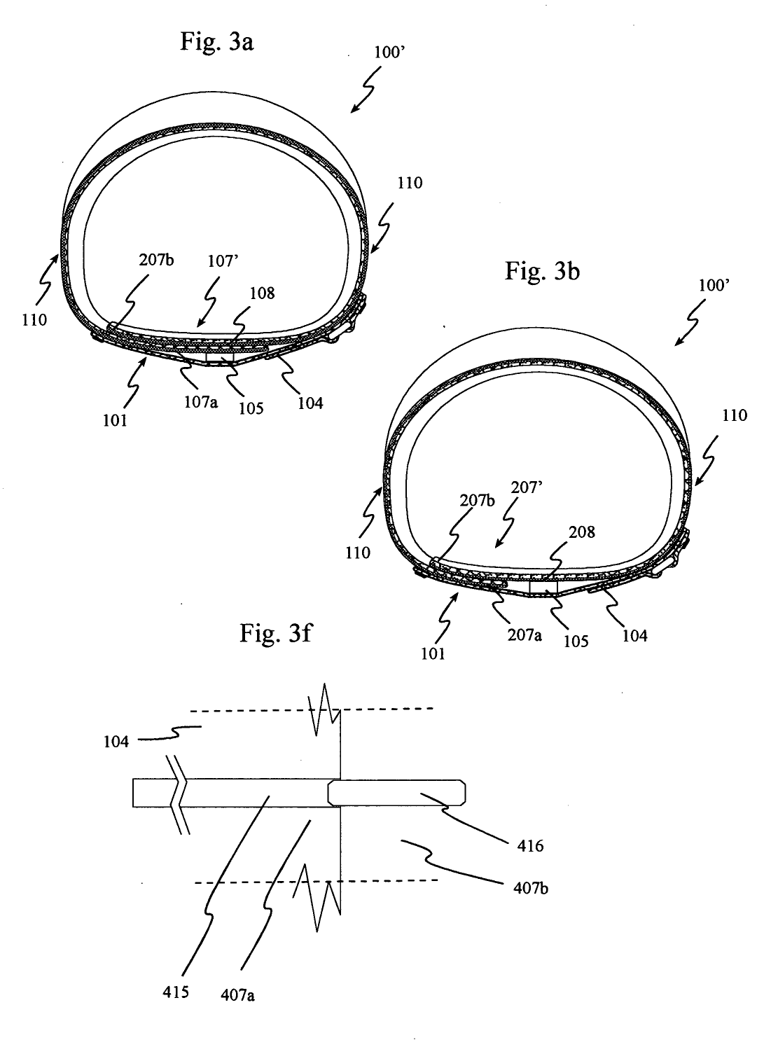

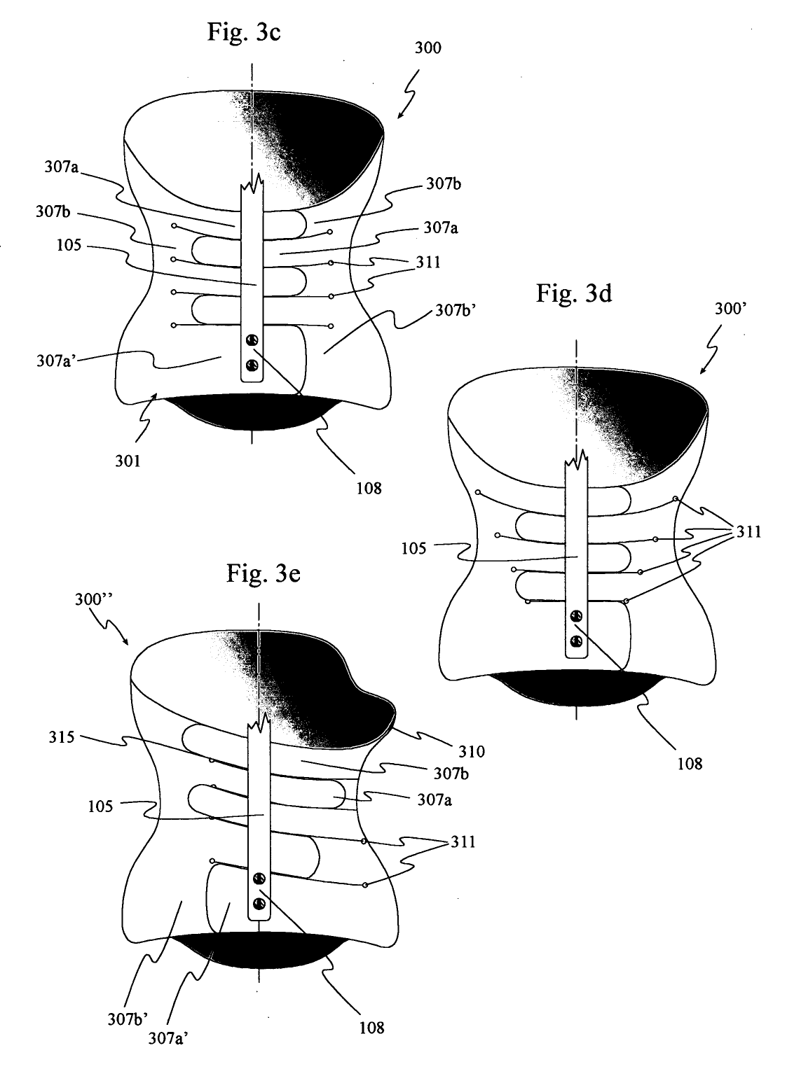

While the above embodiment typifies the present invention the range of embodiments contemplated includes other variations. For example, referring to FIGS. 3a-d an embodiment of the present invention anticipates utilizing a variety of modified FIG. 1 overlap 107 configurations (107′, 207, 307, and 407). FIG. 3a illustrates overlap 107′ in total contact module 100′ which makes use of an extended inner overlap flap 207b flowing entirely across anterior section 101. Extended inner overlap flap 207b places the longitudinal ridge inherently formed by overlapping materials, into lateral transition curve 110 thereby decreasing its unevenness and consequently diminishing the discomfort producing effect to the patients skin. Optionally, extended inner overlap flap 207b is gradually feathered, at a thickness reducing rate that approximates the curvature of lateral transition curve 110, to further reduce the unevenness. Pressure bar 105 is attached at module attachment point 108 on outer overlap flap 107a with, adjustable posterior directed F1 and F2 pressure being applied against anteriorly directed F3 pressure by placement of adjustable straps 104 over pressure bar 105 (See also FIG. 2).

Referring to FIG. 3b, although a sacrifice in rigidity may ensue, a slightly more compact anterior profile is obtainable for total contact module 200 by overlapping extended inner overlap flap 207b with a reduced length outer overlap flap 207a at overlap 207 and placement of a module attachment point 208 for pressure bar 105 on extended inner overlap flap 207b. Strap attachments 106a, on alternating sides at an oppositely balanced distance from pressure bar 105, direct adjustable straps 104 over pressure bar 105 to complementarily positioned strap loops 106b.

Overlaps 107, 107′, and 207 present the inner surface of a single outer overlap flap 107a or 207a contacting the outer surface of a single inner overlap flap 107b or 207b with adjustable straps exerting lateral pressure balanced against the patients abdominal volume thereby stabilizing the relative lateral sliding motion of the one relative to the other. In this configuration, there is no positive registration to prevent outer overlap flap 107a or 207a from sliding longitudinally relative to the position or longitudinal movement of inner overlap flap 107b or 207b causing total contact module 100, 100′, or 200 to become longitudinally or torsionally skewed. In this regard, referring to FIGS. 3c & d, a total contact module 300 interweaves multiple inner and outer overlap flaps 307a and 307b in anterior plastic section 301 including inner and outer overlap flaps 307a′ and 307b′ attaching to pressure bar 105 at module attachment point 108 thereon.

Inner and outer overlap flaps 307a & a′ and 307b & b′ may be formed integrally by lateral shearing cuts of total contact module 300, with each of the shearing cuts preferentially ending with a stress relief hole to prolong material integrity. As inner and outer overlap flaps 307a and 307b slide together longitudinal edge 113 or 114 of each inner overlap flap 307a & a′ interfaces along the entire length of the lateral overlap with the complimentary longitudinal edge 113 or 114 of each of it's longitudinally immediate circumferentially-oppositely-directed neighbors. The longitudinal edges 113 or 114 of each outer overlap flap 307b & b′ likewise interface. Additionally the radially directioned inner surface of each outer overlap flap 307a & a′ interfaces with the respective radially directioned outer surface of the circumferentially oppositely directed inner overlap flap 307b & b′. This arrangement forms a continuous double layer of interlaced inner and outer overlap flaps 307a & a′ and 307b & b′ with freedom of motion laterally (circumferentially) but not longitudinally or torsionally in a skewing motion.

Inner and outer overlap flaps 307a & a′ and 307b & b′ may be of essentially equal lengths or of varying lengths. Referring specifically to FIG. 3c, each inner and outer overlap flaps 307a & a′ and 307b & b′ is essentially the same length. However, referring specifically to FIGS. 3d & e, the multi-flap configuration of total contact module 300 provides a unique opportunity to customize additional specific literally vectored forces to benefit the individual patient. For example as shown here, a pattern of increasing depth can help make the brace more comfortable and effective by making an upwardly directing hydraulic pressure gradient allowing more comfort while at the same time providing no diminishment or an improvement in three point corrective efficiency.

Other means for preventing longitudinal or tortional skewing are also anticipated. Referring to FIG. 3f, an outer overlap flap 407a has one or more registration slots 415 that receive registration guides 416 on an inner overlap flap 407b and prevents longitudinal movement of outer overlap flap 407a relative to inner overlap flap 407b. Strap 104 (dashed lines) crossing at the same level helps keep registration guide 416 engaged within registration slot 415. Also (not shown), module attachment 108 of pressure bar 105 may be a clamp secured to both inner and outer overlap flaps 107a and 107b, with or without relative longitudinal registration, allowing adjustment of the position of module attachment necessitated by progressive change of the medial lateral midplane intersection with inner and outer overlap flaps 107a and 107b.

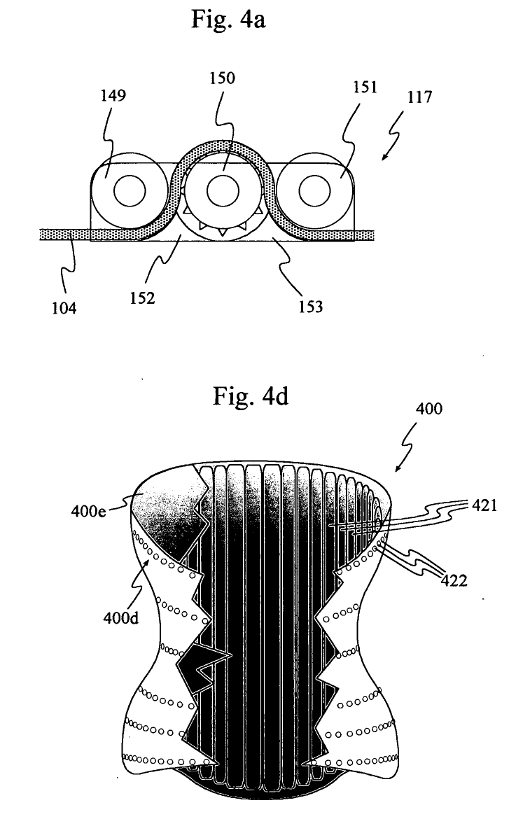

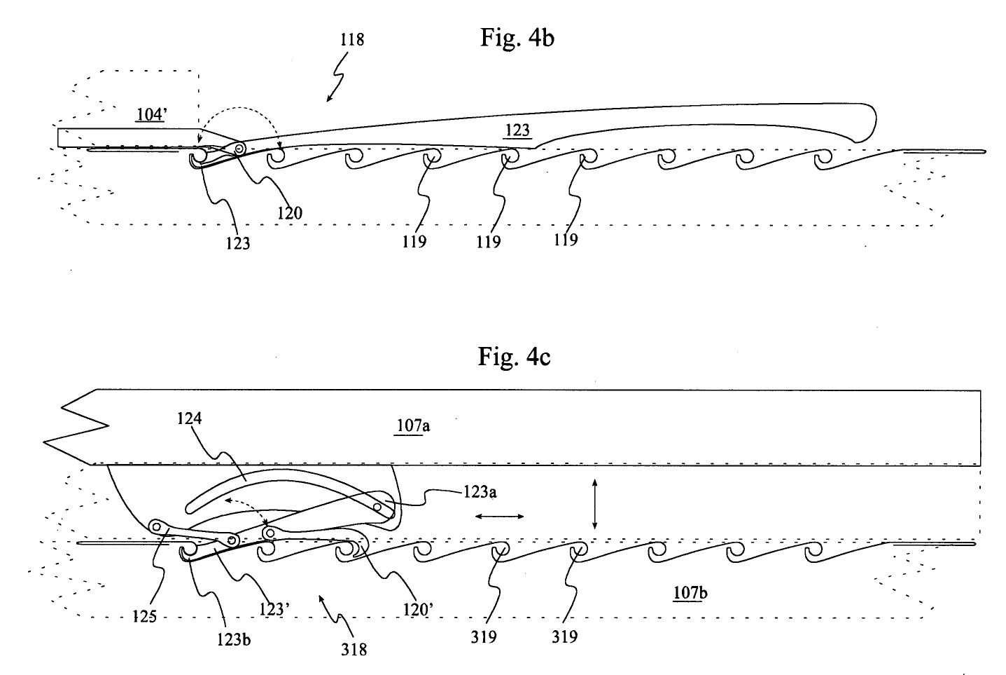

Referring to FIGS. 4a-e, other means for adjusting pressure, than use of adjustable straps 104, are also anticipated as well as situations where adjustment is unnecessary because pressure is fixed. FIG. 4a illustrates a ratcheting take-up mechanism 117 used in place of the FIG. 1 strap loops 106b. Straps 104 are each fed in turn, as ratcheting take-up mechanism 117 is operated, through ratcheting take-up mechanism 117's initial laterally aligned roller pair including an in-feed idle roller 149 and a spiked holding roller 150. An in-feed guide 152 assures that strap 104 is directed between idle roller 149 and spiked holding roller 150. Strap 104 continues over the outside of spiked holding roller 150 and is next fed through a second laterally aligned roller pair including spiked holding roller 150 and an out-feed idle roller 151. An out-feed guide 153 assures that strap 104 will exit ratcheting take-up mechanism 117 without jamming. As ratcheting take-up mechanism 117 further operates, strap 104 tightens. The ratchet gear and handle including pawls, being common, are not shown in this figure.

Referring to FIG. 4b, another alternative anticipated would be substitution of walking clasps 118 for pulling tensioning straps 104 through strap loops 106b. Here a single action walking clasp 118 is utilized as shown to pull the end of tensioning strap 104′, or the edge of outer overlap flap 107a, along a series of linearly spaced hook notches 119, incrementally tightening tensioning strap 104′ in alternating series with all other tensioning straps 104′. A pawl 120 keeps walking clasp 118 engaged with one hook notch 119 while an anchor handle 123 advances to the next hook notch 119.

Referring to FIG. 4c in the context of FIGS. 3d-e, double action walking clasps 318′ are integrated onto the inner surfaces of the plastic shell layer of outer overlap flaps 307a, and linearly spaced hook notches 319 are aligned therewith onto the outer facing surfaces of inner overlap flaps 307b. Each walking clasp 318 is advanced sequentially along hook notches 319 by simply lifting outer overlap flap 307a away from inner overlap flap 307b then pressing outer overlap flap 307a back toward inner overlap flap 307b to gradually tighten total contact module 300. As outer overlap flap 307a separates from inner overlap flap 307b, an arcuate slot 124 engaged sliding attachment end 123a of anchor handle 123 rotates against engaged notch 119 of a pawl 120′ advancing a drag bar 125 a distance of one-half notch 119 while at the same time advancing an anchor end 123b of anchor handle 123 to its next notch 119. Then pressing outer overlap flap 307a back toward inner overlap flap 307b causes sliding attachment end 123a of anchor handle 123 to rotate against its engagement with notch 119 further advancing drag bar 125 one half notch while at the same time advancing pawl 120′ to its next notch 119. Once adjusted sufficiently, outer overlap flaps 307a are immobilized, for example by hook and loop attachment material (Velcro™). This embodiment has eliminated the need for straps as a means to tighten total contact module 300 by making the outer overlap flaps 307a function as straps. Consequently, in this embodiment pressure bar 105 must pass between one or more outer overlap flap 307a and inner overlap flap 307b. Therefore, only the smooth outer surface of the total contact module is exposed, eliminating module surface clutter thereby decreasing intrusion on the patients appearance.

The present invention must be worn snugly in order for it to be most effective. In instances where compliance with this snugness requirement is problematic, a non-patient-adjustable device may be desirable. However, the device must remain adjustable by the orthotist so that it can be easily reconfigured each time the patient visits the office. Referring to FIG. 4d a multiple flap total contact module 400 has one position clasps 418 and single hook notches 419 (not shown). However, one or more rubber bladders 434 intercede between plastic shell 400d and resilient lining 400e. Injection ports 422 in shell 400d communicate with the interior of each bladder 421 allowing the orthotist to adjust the pressure of each bladder 421 by injecting or removing a compression gel, fluid, or gas. This not only helps with compliance but gives the orthotist the opportunity to specifically adjust the exact points of pressure as the patient's body begins to respond over time to the influence of the brace.

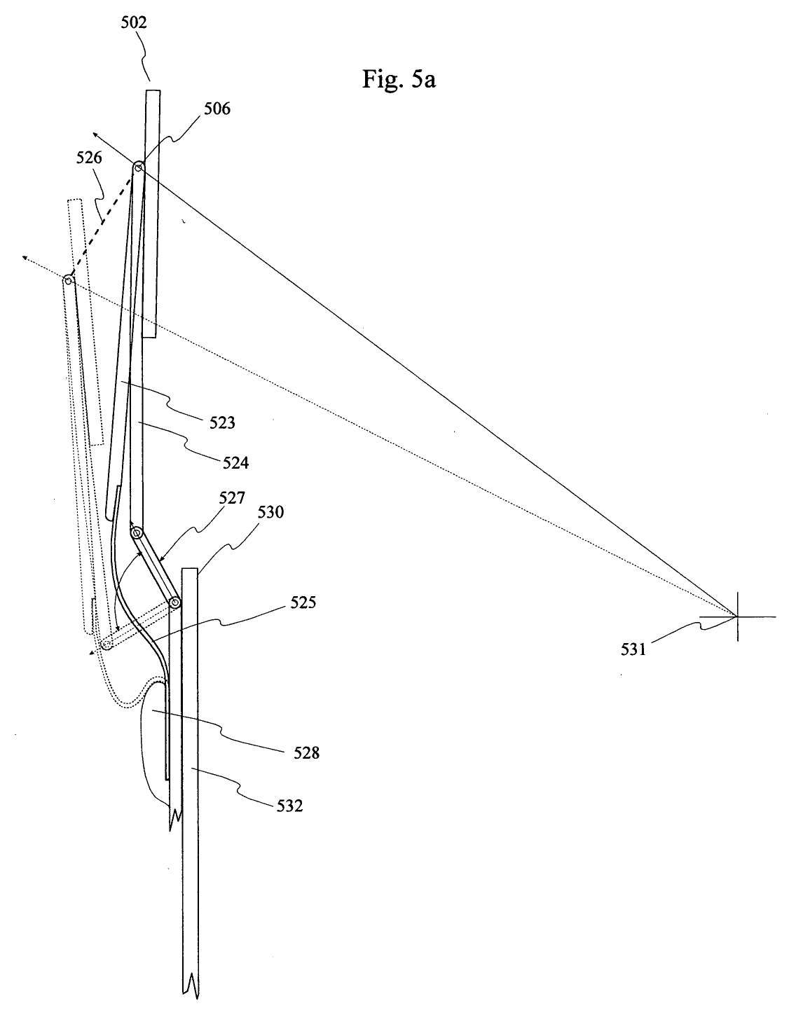

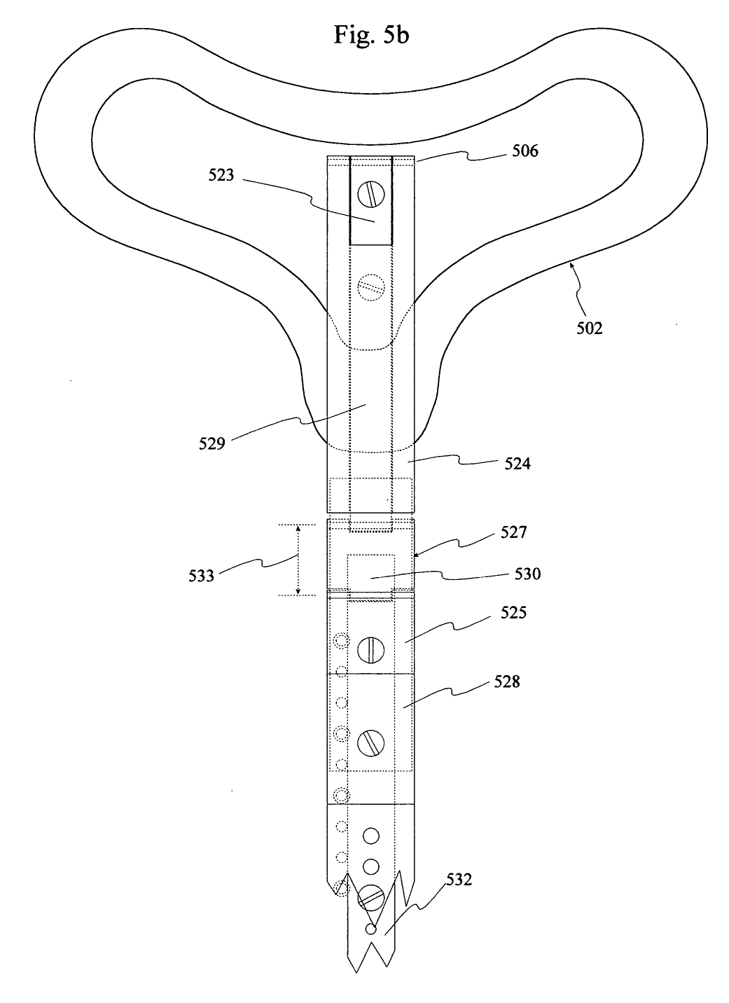

The present invention also anticipates, for another example, alternatives in the pressure bar. Since the fitting of an orthotic often results in the patient becoming more ambulatory, such alternatives could include auto-compensating features to allow more appropriate support as the patient moves from one position to another (such as standing to sitting) during normal activities. Referring to FIGS. 5a-b and 6a-i a pressure bar 505 at the total contact module 500 distal end (The herein terminology “module distal” or “module proximal” indicates position relative to the total contact module and “plate distal” or “plate proximal” indicates position relative to the chest plate), includes a single pivot hinge 506 just module distal beyond chest plate attachment point 509 rotatably connecting chest plate pivot restriction bar 523 with the module distal end of a pivot support bar 524. The module proximal end of pivot support bar 524 is fixed to the module distal end of an S-shaped spring 525. The module proximal end of S-shaped spring 525 is adjustably fixed to the module distal end of chest plate adjustment bar 523 by the clamping action of a spiral spring intensifier block 528 fastened to chest plate adjustment bar 523 at the same position. A double pivot axis translating hinge 527 within the length of chest plate adjustment bar 523 translates an axis of movement 531 of chest plate 502 posteriorly and module proximally such that a path of deflection 526, thereof, approaches the natural path that the chest follows during sitting-standing transitions.

A channel 528 of increasing depth in pivot support bar 524 accommodates complete closure against chest plate pivot restriction bar 523 defining one limit of rotation of chest plate 502 about single pivot hinge 506. Oppositely, an extension 530 of a module attachment bar section 532 defines the other limit of rotation of chest plate 502 about single pivot hinge 506. An axis separation distance 533 of double pivot hinge is empirically defined as that measure that will allow path of deflection 526 to accommodate the foreshortening of the patient's trunk anterior side that occurs as a result of sitting.

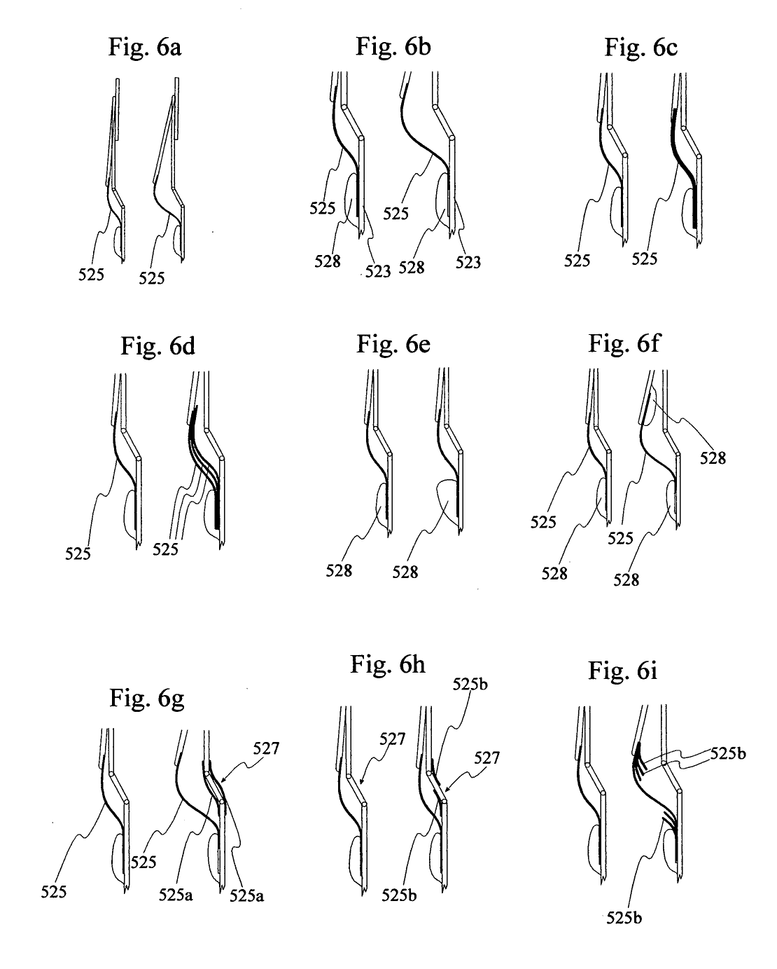

Spiral spring intensifier block 528 positioned in contact with S-shaped spring 525 at a spirally curved surface 528a decreases the length of S-shaped spring 525 as it's deflection increases thereby providing an increasing resistance to increasing deflection of chest plate 502. The limits and range of pressure intensity exerted by S-shaped spring 525 can be adjusted by one or more of:
A) changing the length of S-shaped spring 525 (FIG. 6a);
B) changing the amount of S-shaped spring 525 that is clamped between spiral spring intensifier block 528 and chest plate adjustment bar 523 (FIG. 6b);
C) increasing the thickness of S-shaped spring 525 (FIG. 6c);
D) deploying multiple S-shaped springs 525 sandwiched into the same position (FIG. 6d);
E) changing the thickness of spiral spring intensifier block 528 (FIG. 6e);
F) adding a second spiral spring intensifier block 528 at the module distal end of S-shaped spring 525 (FIG. 6f);
G) adding second or third S-shaped springs 525a spanning double pivot axis translating hinge 527 (FIG. 6g);
H) adding one or more helper springs 525b to one or both ends of S-shaped spring 525 (FIG. 6h);
I) adding one or more helper springs 525b to one or both of the pivots of double pivot axis translating hinge 527 (FIG. 6i); etc.
   this ability to adjust range and intensity of spring pressure allows the orthotist the capability of building an F1−F3−F2 force-differential hyperextension-pressure component that is balanced specifically to the size and weight of the patient and well as to the targeted amount of hyperextension. Additionally, the plate distal remainder of pressure bar 505 can be more rigidly attached across the entire longitudinal length of total contact module 500. In this case, adjustment of abdominal hydraulic pressure component exerted by the enclosure by total contact module 500 of the abdominal region is independent of the intensity of the F1−F3−F2 force-differential hyperextension-pressure component.

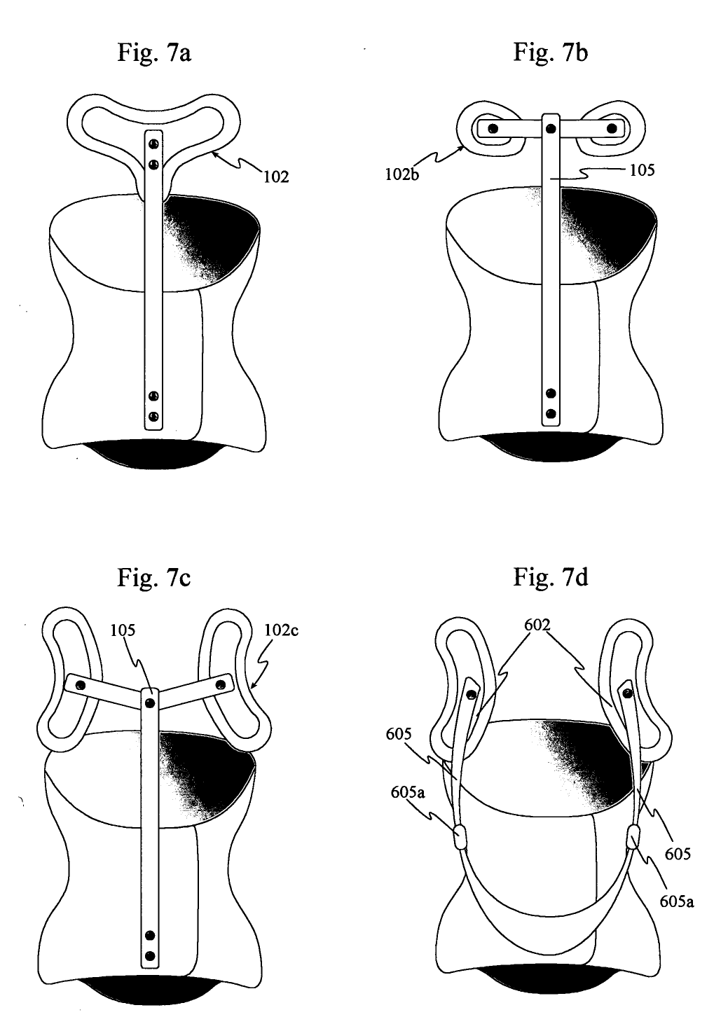

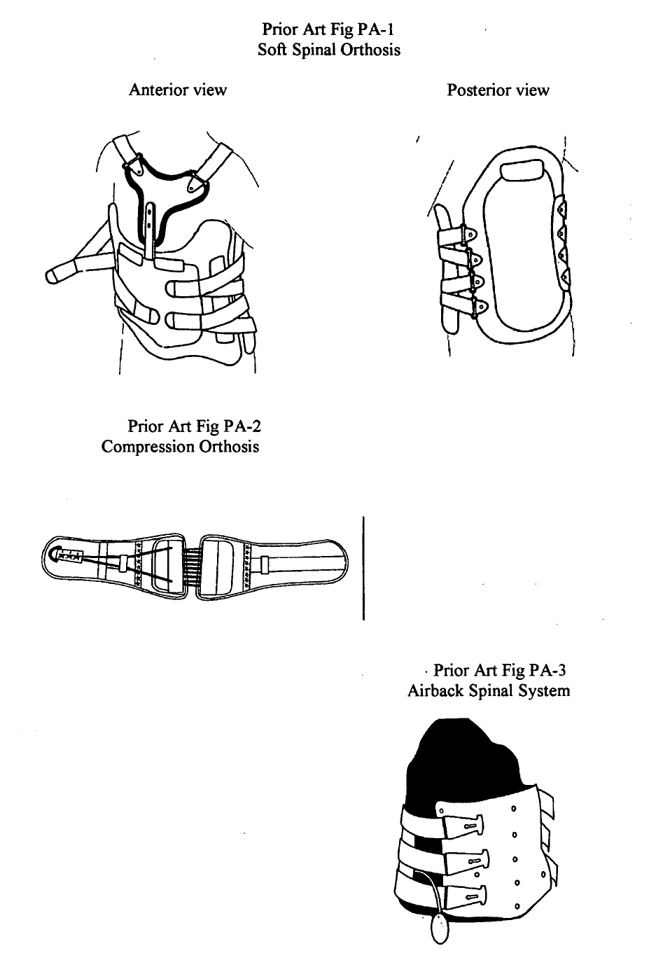

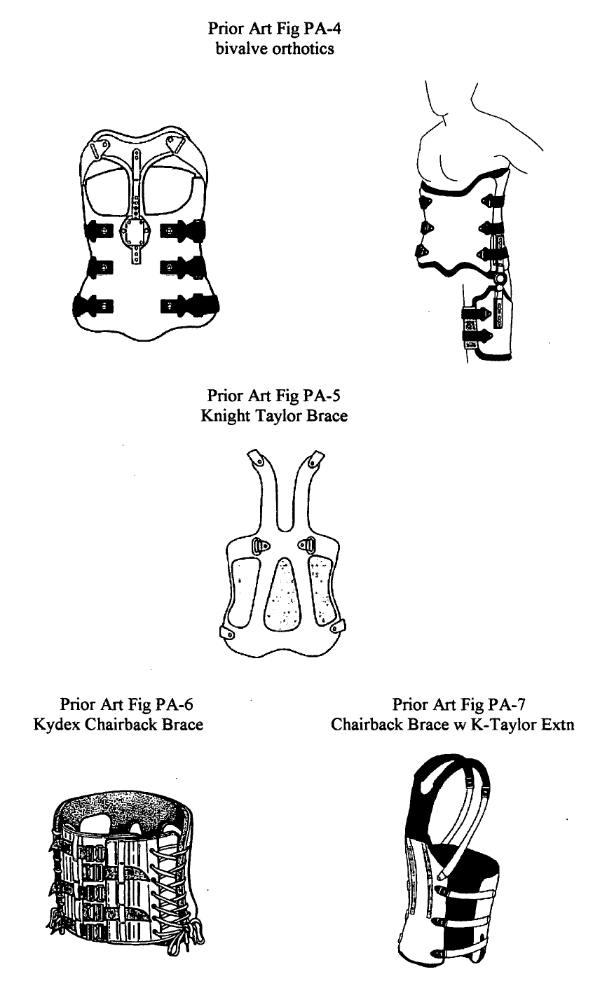

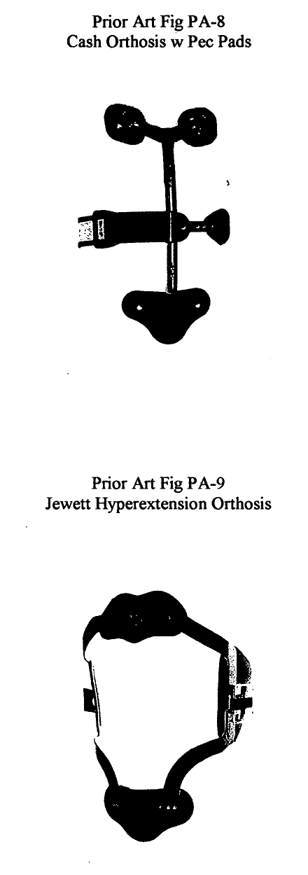

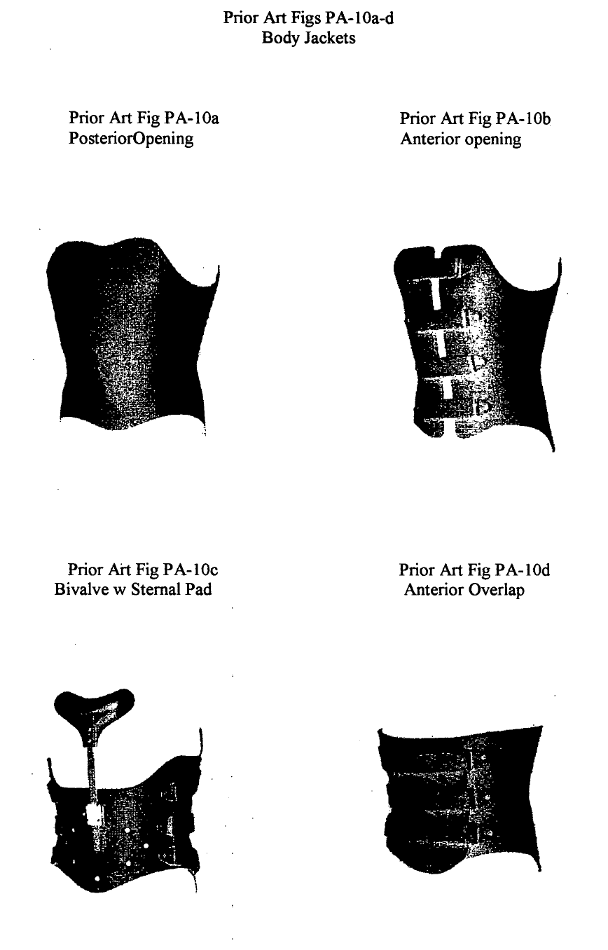

Referring to FIGS. 7a-d the scope of chest plate 102 utilization is not limited by the previously shown embodiments. FIG. 7a illustrates the now familiar sternal plate utilized in the preceding illustrations. However it is anticipated that any of the many configurations of upper body attachments are adaptable to the present invention. For example, FIG. 7b illustrates the present invention with a pectoral pad bridge module 102b attached to pressure bar 105, while FIG. 7c illustrates using a shoulder pad bridge module attached to pressure bar 105, and FIG. 7d even illustrates shoulder pads 602 on lateral cantilevered pressure bars 605 with snap-in-place resilient pivots 605a and with or without outward tortional bias. Full cervical braces are also contemplated as well as pelvic and leg augmentation attachments or integrated extensions.

Esthetic improvements to braces are also anticipated by this invention, including but not limited to light weight due to improvement in materials, as well as decreased intrusion on the patients appearance due to the low profile mechanics, and less cluttered exterior surfaces. Manufacturing advantages are also anticipated including the use of materials that are easily workable, as well as modular attachment features.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defined in the appended claims.

What is claimed is:

1. A spinal orthotic, comprising:
a total contact module constructed of a single piece of a semi-rigid semi-resilient material, said total contact module having an anterior overlap;
a longitudinal pressure bar positioned anterior to said total contact module, said longitudinal pressure bar spanning first and second posteriorly directed force vectors;
a module attachment at said first posteriorly directed force vector, said module attachment attaching a module attachment end of said longitudinal pressure bar to said total contact module;
a chest plate attachment at said second posteriorly directed force vector, said chest plate attachment attaching a chest plate attachment end of said longitudinal pressure bar to a chest plate;
at least one adjustable strap on said module spanning said anterior overlap;
at least one of said at least one adjustable strap being capable of increasing said anterior overlap to reduce a circumferential size of said total contact module thereby increasing abdominal hydraulic pressure;
at least one of said at least one adjustable strap being capable of inducing an anteriorly directed third force parallel with, between, and diametrically opposite said first and second forces by connecting a tenth thoracic vertebrae proximal region on a posterior side of said total contact module with a posterior pressure directing anchor point on said longitudinal pressure bar between said chest plate attachment end and said module attachment end; and,
said first, second, and third forces collectively forming a three point pressure system having a spinal extension directing effect.

2. The spinal orthotic of claim 1, further comprising:
said semi-rigid semi-resilient material being one of a homogeneous material and a laminate;
said laminate having at least one of a shell and a liner;
said shell at least partially having at least one layer of at least one of a clothing contact surface material, a core, a stiffener, and a strengthening material; and,
said liner at least partially having at least one layer of at least one of a resilient cushion and a dermal contact surface layer.

3. The spinal orthotic of claim 2, wherein:
said clothing contact surface material is selected from the group consisting of a plastic, a metal, an alloy, a cloth, a leather, a rubber, a polyethylene, a polypropylene, a polyvinylchloride, a polybuterate, a polystyrene, a polycarbonate, and an aluminum;
said core is selected from the group consisting of a plastic, a metal, an alloy, a cloth, a leather, a rubber, a polyethylene, a polypropylene, a polyvinylchloride, a polybuterate, a polystyrene, a polycarbonate, and an aluminum;
said strengthening material is selected from the group consisting of a plastic, a metal, an alloy, carbon fibers, glass fibers, plastic fibers, a cloth, a leather, a rubber, a polyethylene, a polypropylene, a polyvinylchloride, a polybuterate, a polystyrene, a polycarbonate, and an aluminum;
said stiffener is selected from the group consisting of a plastic, a metal, an alloy, a cloth, a leather, a rubber, a polyethylene, a polypropylene, a polyvinylchloride, a polybuterate, a polystyrene, a polycarbonate, and an aluminum;
said resilient cushion is selected from the group consisting of a plastic foam, a cloth, a leather, a rubber foam, a polyethylene foam, a polypropylene foam, a polyvinylchloride foam, a polybuterate foam; and
said dermal contact surface material is selected from the group consisting of a plastic, a cloth, a leather, a rubber, a polyethylene, a polypropylene, a polyvinylchloride, a polybuterate, a polystyrene, and a polycarbonate.

4. The spinal orthotic of claim 1, further comprising:
said overlap includes at least one inner flap and at least one outer flap;
both said inner and outer flaps extend an essentially equivalent distance past a sagittal anterior-posterior midplane; and,
said module attachment end of said longitudinal pressure bar attaches to one of said at least one outer flap at said sagittal anterior-posterior midplane.

5. The spinal orthotic of claim 1, further comprising:
said overlap includes at least one inner flap and at least one outer flap;
said inner flap essentially extends completely across an anterior side of said total contact module; and,
said outer flap extends a sufficient distance past a sagittal anterior-posterior midplane to allow said module attachment end of said longitudinal pressure bar to attach to one of said at least one outer flap at said sagittal anterior-posterior midplane.

6. The spinal orthotic of claim 1, further comprising:
said overlap includes at least one inner flap and at least one outer flap;
said inner flap essentially extends completely across an anterior side of said total contact module;
said outer flap extends a sufficient distance across an anterior side to create said overlap; and,
said module attachment end of said longitudinal pressure bar attaches to one of said at least one inner flap at a sagittal anterior-posterior midplane.

7. The spinal orthotic of claim 1, further comprising:
said overlap includes first and second circumferential end margins of said single piece divided into multiple overlappable flap sections serially positioned, with each touching the next, along each lengths of said first and second circumferential end margins;
each said overlappable flap section of said first circumferential end margin pairs with a longitudinally aligned one of said overlappable flap sections of said second circumferential end margin as an overlap pair;
some said overlappable flap sections of said first circumferential end margin are outer flaps paired with said overlappable flap sections of said second circumferential end margin as inner flaps; and,
the remainder of said overlappable flap sections of said first circumferential end margin are inner flaps paired with said overlappable flap sections of said second circumferential end margin as an outer flaps.

8. The spinal orthotic of claim 1, wherein said chest module is one of a chest plate, a bridged pectoral pad set, a bridged shoulder pad set, a cantilevered shoulder pad set, and a cantilevered shoulder pad set having outward tortional bias.

9. The spinal orthotic of claim 8, wherein said chest module further comprises a cervical support feature.

10. The spinal orthotic of claim 1, wherein at least one of said module attachment and said chest plate attachment is a hinge having an axis of rotation perpendicular to a sagittal anterior-posterior midplane.

11. The spinal orthotic of claim 10, further comprising:
said hinge includes at least one additional axis of rotation;
each said additional axis of rotation is parallel to said axis of rotation;
collectively all said axes of rotation defining a compound hinge;
said compound hinge including a resilient element; and
said resilient element urging said at least one of said module attachment and said chest plate attachment in a posterior direction against said posterior pressure directing anchor point.

12. The spinal orthotic of claim 7, further comprising:
said longitudinal pressure bar passing between said outer and inner flaps of at least one said overlap pair;
one said outer flap of said at least one said overlap pair having an adjustable latch whereby said one said outer flap can attach to said total contact module and exert a size reducing pressure on said circumferential size; and
said one said outer flap is aligned over said posterior pressure directing anchor point and replaces said at least one of said at least one adjustable straps being capable of inducing an anteriorly directed third force.

13. The spinal orthotic of claim 10, further comprising:
at least one of said module attachment and said chest plate attachment is a hinge having an axis of rotation perpendicular to a sagittal anterior-posterior midplane;
said hinge includes at least one additional axis of rotation;
each said additional axis of rotation is parallel to said axis of rotation;
collectively all said axes of rotation defining a compound hinge;
said compound hinge including a resilient element; and
said resilient element urging said at least one of said module attachment and said chest plate attachment in a posterior direction against said posterior pressure directing anchor point.

14. An orthosis, comprising,
a one piece abdominal enclosure having an anterior overlap, said one piece abdominal enclosure capable of exerting an abdominal hydraulic pressure; and
a chest plate attached to said anterior overlap by a pressure bar whereby said one piece abdominal enclosure, said chest plate, and said pressure bar collectively in concert form a three point spinal hyperextension pressure system.

15. The orthosis of claim 14, further comprising:
said anterior overlap including at least one outer and inner overlap flap pair;
an axis translating compound hinge having an adjustable resilient element with at least one adjustment mechanism, said resilient element biasing said chest plate posteriorly and said axis translating compound hinge integrated into said pressure bar at a module distal portion thereof

16. The orthosis of claim 14, further comprising:
at least one internal bladder disposed on an interior surface of said one piece abdominal enclosure; and
position fixing latches connecting said at least one outer and inner overlap flap pair to a relative fixed position.

17. The orthosis of claim 15, further comprising:
at least one internal bladder disposed on an interior surface of said one piece abdominal enclosure;
an access port in said one piece abdominal enclosure for each said at least one internal bladder; and
position fixing latches connecting said at least one outer and inner overlap flap pair to a relative fixed position.

18. The orthosis of claim 15, wherein said at least one adjustment mechanism is selected from the group consisting of:
changing a length of an S-shaped spring;
changing the amount of said S-shaped spring that is clamped between a spiral spring intensifier block and a chest plate adjustment bar;
increasing a thickness of said S-shaped spring;
deploying multiple said S-shaped springs sandwiched together;
changing the thickness of said spiral spring intensifier block;
adding additional said spiral spring intensifier blocks;
adding second or third S-shaped springs spanning a double pivot axis translating hinge;
adding one or more helper springs to one or both ends of said S-shaped spring; and
adding one or more helper springs to one or both pivots of said double pivot axis translating hinge.

19. The orthosis of claim 17, wherein said at least one adjustment mechanism is selected from the group consisting of:
changing a length of an S-shaped spring;
changing the amount of said S-shaped spring that is clamped between a spiral spring intensifier block and a chest plate adjustment bar;
increasing a thickness of said S-shaped spring;
deploying multiple said S-shaped springs sandwiched together;
changing the thickness of said spiral spring intensifier block;
adding additional said spiral spring intensifier blocks;
adding second or third S-shaped springs spanning a double pivot axis translating hinge;
adding one or more helper springs to one or both ends of said S-shaped spring; and
adding one or more helper springs to one or both pivots of said double pivot axis translating hinge.
